(19) United States
(12) Patent Application Publication
(10) Pub. No.: US 2026/0192075 A1
(43) Pub. Date: Jul. 9, 2026

(54) CONNECTOR MEANS, TAPE, TRACHEAL CANNULA AND USE OF A CONNECTOR MEANS

(71) Applicant: ANDREAS FAHL MEDIZINTECH-NIK-VERTRIEB GMBH, Köln (DE)

(72) Inventor: Andreas Fahl, Köln (DE)

(73) Assignee: ANDREAS FAHL MEDIZINTECH-NIK-VERTRIEB GMBH, Köln (DE)

(21) Appl. No.: 19/558,913

(22) Filed: Mar. 6, 2026

Related U.S. Application Data
(63) Continuation of application No. PCT/EP2024/074921, filed on Sep. 6, 2024.

(30) Foreign Application Priority Data
Sep. 7, 2023 (DE) 10 2023 124 092.7

(51) Int. Cl. A61M 16/08; A61M 16/04

(52) U.S. Cl. CPC A61M 16/0816; A61M 16/047; A61M 2205/0216

(57) ABSTRACT

What is proposed is a connector for connection of a tracheostoma aid, wherein the connector includes a connector ring. The connector ring includes a first snap ring and at least one second snap ring. The first snap ring and the second snap ring are formed on an inner surface of the connector ring. The first snap ring and the second snap ring have different inner diameters.

### CROSS-REFERENCE TO RELATED APPLICATIONS

This Application is a continuation of International Application No. PCT/EP2024/074921, filed on Sep. 6, 2024, which in turn claims priority to German Application No. 1020231240927, filed on Sep. 7, 2023, the entire contents of both incorporated herein by reference in their entirety.

### TECHNICAL FIELD

The present disclosure relates to a connector, a tape, a tracheal cannula and a use of a connector.

### BACKGROUND

Connectors for tracheostoma aids are well known from the prior art. EP 2 953 674 B1 discloses a tracheostoma tape with a receptacle for a tracheostoma aid, such as a moisture exchanger or a speaking valve, wherein the tracheostoma aid can be fastened in the receptacle by a clamp connection.

### SUMMARY

It is an object of the present disclosure to provide an improved connector. It is an object of the present disclosure to provide a connector, which securely holds a tracheostoma aid. It is further an object of the present disclosure to provide a connector by which a large number of different tracheostoma aids can be received.

The object is solved according to the present disclosure by a connector for connection to a tracheostoma aid, wherein the connector includes a connector ring, wherein the connector ring includes a first snap ring and at least one second snap ring, wherein the first snap ring and the second snap ring are formed on an inner surface of the connector ring and wherein the first snap ring has a first inner diameter and the second snap ring has a second inner diameter, wherein the first inner diameter is larger than the second inner diameter, wherein the first snap ring is arranged distally from the second snap ring.

The object is furthermore solved according to the present disclosure by a tape with an above-mentioned connector, wherein a connecting piece is arranged on a distal side of the tape.

The object is furthermore solved according to the present disclosure by a tracheal cannula with an above-mentioned connector, wherein a connector ring is arranged on a distal end of a cannula tube.

The object is furthermore solved according to the present disclosure by a use of a connector as mentioned above for connection to tracheostoma aids with different outer diameters.

A connector for connection of a tracheostoma aid is proposed, wherein the connector includes a connector ring. The connector ring includes a first snap ring and at least one second snap ring. The first snap ring and the second snap ring are formed on an inner surface of the connector ring. The first snap ring and the second snap ring have different inner diameters.

The application of an artificial breathing opening, the tracheostoma, in the trachea can be necessary during surgical procedures in the upper respiratory tract, so that air can be inhaled directly into the lung by bypassing oral cavity and larynx. In laryngectomized individuals, thus individuals without larynx, whose larynx was surgically removed, the tracheostoma has to be kept open and stabilized permanently, for the purpose of which tracheal cannulas, generally consisting of an outer and an inner cannula, are inserted into the tracheostoma. However, the use of so-called “tracheostoma buttons” is also possible, for patients who otherwise no longer need a tracheal cannula.

So-called “shunt valves” can furthermore also be inserted into the tracheostoma, which make it possible to restore the voice. Finally, filter systems can also be inserted into the tracheostoma, both in tracheostomized individuals and in laryngectomized individuals. Filter systems of this type either consist of a tape with an inserted filter or of a usually self-adhesive connecting piece or a base plate, respectively, generally made of plastic, into which filters of all kinds can be inserted.

The so-called “moisture and heat exchangers”, also referred to as “artificial nose”, are among the filter systems, which are used for laryngo-tracheal aids, such as tracheal cannulas, shunt valves, tracheostoma buttons and filter systems with tape or base plate. They serve the purpose of replicating the regulatory mechanisms missing in laryngectomized and also tracheotomized patients for heating up and moistening the breathing air and of avoiding bringing the trachea into contact with increased dry, cold and unfiltered air. This is so because an increased mucus production with the subsequent risk of incrustation occurs due to the irritation caused hereby. The breathed-in air is now moistened, heated up and simultaneously filtered by moisture and heat exchangers. The above-mentioned crust formation is largely avoided hereby. Routinely wearing the artificial nose in particular in response to heavy secretion helps thereby because the discharge production is reduced by moistening the mucous membranes in the trachea.

Moisture and heat exchangers for laryngectomized and tracheotomized individuals have a filter body, generally made of paper or foam material, through which the air, which is breathed in and breathed out, is guided. When breathing out, the filter body retains moisture, which is then transported into the trachea when breathing in. Artificial noses known from the prior art, also referred to as “HME” (humid and moisture exchanger), are available in a large variety of different designs for different adapters. The universal adapter according to DIN EN ISO 5356-1:2015-06 thereby has a diameter of 15 mm and 22 mm, but further adapters with in particular larger diameters, for example 16 mm and 23 mm, are also known from the prior art. The universal adapter of, for example, 15 mm or 16 mm, is used in particular in the case of tracheal cannulas, whereas tracheostoma buttons and filter systems, in particular formed with a self-adhesive base plate or with a tape, have a larger adapter diameter, such as, for example, 22 mm or 23 mm.

A tracheostoma aid can be, for example, a heat-moisture exchanger or a speaking valve or a combination of both. It is provided in one design that the tracheostoma aid, which can be received by the connector, in some embodiments, has an outer diameter of approximately 11 mm to approximately 25 mm, in other embodiments, approximately 21 mm to approximately 24 mm, in other embodiments, approximately 11.5 mm, in other embodiments approximately 15 mm, in other embodiments approximately 16 mm, in other embodiments approximately 22 mm, in other embodiments approximately 23 mm, at least in sections.

Exemplary enumerations, such as the ones above, are not to be considered to be exhaustive in terms of the present disclosure but can be supplemented in the context of the general expert knowledge.

It is an advantage of the proposed connector that the latter can receive tracheostoma aids of a different size by means of the at least two snap rings. The connector can in particular receive at least one standardized tracheostoma aid and at least one further, non-standardized tracheostoma aid.

The connector includes a proximal connecting piece. It is provided in one aspect that the connector includes a connecting piece, which is formed as a base plate. In an embodiment, the connecting or the base plate, respectively, is an annular disk, which is arranged on the proximal end of the connector ring on the outer wall thereof. The connecting extends radially outwards on the proximal end of the connector ring. An adhesive, for example an adhesive film, such as with a peelable protective film, is arranged proximally on the connecting or the base plate, respectively. Connector with adhesive then form a tracheostoma tape, for example.

It is provided in a further aspect that a cannula is arranged proximally from the connector ring. For example, the cannula is a cannula tube. For example, the connector and cannula tube form a tracheostoma button or a tracheal cannula.

If directional information is used in the context of the description of the present disclosure, this information is to be understood with regard to the customary use of the connector. Directional terms are used as is customary in anatomy. In terms of the present disclosure with regard to a feature of the device according to the present disclosure, the term “distal” is understood to be an arrangement or use thereof, respectively, away from or also facing away from or lying opposite a tracheostoma tape or a tracheal cannula, more generally a skin surface of an individual, who wears such fastening for the device according to the present disclosure. Distal is to be understood to be a direction away from the body of the individual. In terms of the present disclosure with regard to a feature of the device according to the present disclosure, the term “proximal” is understood to be an arrangement or use thereof close to or also facing or adjacent to a tracheostoma tape or a tracheal cannula, more generally a skin surface of an individual, who wears such fastening for the device according to the present disclosure. Proximal is to be understood to be a direction towards the body of the individual.

The connector includes a connector ring. The connector ring is cylindrical. The connector ring includes an outer jacket. The connector ring includes an inner surface. The inner surface extends from a proximal end of the connector ring to a distal end of the connector ring on the inner side thereof. It is provided in one aspect that the connecting piece is arranged on a proximal side of the connector ring.

In one embodiment, the connector, such as the connecting piece and/or the connector ring, includes a material selected from a group including silicone, thermoplastic elastomers and/or polyvinyl chloride. In one embodiment, the connector ring is made of the same material as the connecting piece. In a further embodiment, the connector ring is material-connected to the connecting piece. For example, the connector ring and the connecting piece is made in a one- or two-component injection molding.

The connector ring includes a first snap ring and at least one second snap ring. In terms of the present disclosure, a snap ring is understood to be a first part of a snap connection. The snap connection includes functional elements for the releasable and non-releasable, simple positive connecting of joining parts, in particular of tracheostoma aids to the connector. At least a portion of the joining parts deforms elastically thereby and subsequently interlocks, releasably or non-releasably. At least the connector ring, the snap ring and/or at least a portion of the tracheostoma aid is elastically deformable. The first and/or second snap ring can be formed in a completely circumferential manner in the connector ring. In one embodiment, the first and/or second snap ring is formed in segments. The first and/or second snap ring is formed in a circumferential manner in sections in the connector ring. First and/or second snap ring and tracheostoma aid can be hooked together. The snap connection of tracheostoma aid and first and/or second snap ring is releasable. Tracheostoma aid and connector can be joined in a positive manner. The connector ring forms a ring snap connection with the tracheostoma aid. The connector ring is formed as snap cylinder. It is provided in one design that the connector ring has three or more than three snap rings. A plurality of different tracheostoma aids can be received therewith.

It is an advantage of a snap connection between tracheostoma aid and connector that the parts can be easily joined together. When joined, however, the parts are in particular firmly connected to one another in a positive manner in such a way that the tracheostoma aid does not fall out of the connector during use.

It is provided in one aspect that the connector ring has a first material thickness between the first snap ring and the second snap ring and has a second material thickness proximally from the second snap ring. The functionality is hereby ensured with simultaneously sufficient stability.

In some aspects of the tracheostoma aid, the latter does not include a suitable undercut for a snap connection with the first or second snap ring. The snap ring acts as clamping ring in this case. It is provided in one embodiment that the tracheostoma aid can be fastened in a clamping manner by the first or the second snap ring, so that a clamp connection is provided. Depending on the dimensioning or depending on the geometric embodiment of the tracheostoma aid, respectively, the latter is held by the first or second snap ring by a clamp connection or a snap connection.

In terms of the present disclosure, undercut of the tracheostoma aid is to be understood in such a way that a diameter of the tracheostoma aid or of the conical connector thereof, respectively, becomes smaller in sections in the opposite direction of the joining direction, so that a snap ring can engage behind a section of the tracheostoma aid or of the conical connector, respectively.

In one embodiment, the first and/or second snap ring include at least one beveled distal and/or proximal sliding surface. In a longitudinal section of the connector ring, the sliding surface has, in some embodiments, an angle of approximately 30° to approximately 50°, in other embodiments, approximately 38° to approximately 48°, and in other embodiments, approximately 45° to a center line, which extends in the direction of extension of the connector ring. The tracheostoma aid can advantageously be easily inserted into the connector ring by the sliding surface. It is provided in one design that a sliding surface is provided on the distal side of the first and/or second snap ring, wherein the proximal side of the first and/or second snap ring preferably does not have a beveled distal and/or proximal sliding surface. The first and/or second snap ring includes a distal surface, which extends essentially in a plane perpendicular to a central axis of the connector ring. An insertion of the tracheostoma aid into the connector is advantageously simplified hereby, wherein the tracheostoma aid cannot be removed easily or cannot fall out, respectively, after the insertion and the snap-in downstream from the first and/or second snap ring, so that a connection of the two parts takes place.

When the term “approximately” is used in the context of the present disclosure in connection with values or value ranges, this is to be understood to be a tolerance range, which the person of skill in the art considers to be customary, in some embodiments a tolerance range of ±20%, in other embodiments ±10%, and/or in other embodiments ±5% is provided. If different value ranges, for example preferred and more preferred value ranges, are specified in the present disclosure, the lower limits and the upper limits of the different value ranges can be combined with one another.

In one embodiment, only the second snap ring includes a sliding surface. In one embodiment, only the first snap ring includes a sliding surface.

It is provided in one aspect that the first snap ring has a first maximum material thickness and the second snap ring has a second maximum material thickness, wherein the first maximum material thickness is smaller than the second maximum material thickness. A first minimum material thickness of the first snap ring is preferably approximately identical to a second minimum material thickness of the second snap ring. The first and/or the second snap ring includes a minimum material thickness essentially on a ring inner edge located radially on the inside. The first and/or the second snap ring includes a maximum material thickness on the side facing the inner surface of the connector ring.

A sliding surface advantageously provides for a simple joining of the tracheostoma aid into the connector ring. A surface of the tracheostoma aid, such as a portion of a snap connection of the tracheostoma aid, can slide easily over the sliding surface in this way and can then lock in place downstream of the first and/or second snap ring in the joining direction. Even if the tracheostoma aid does not have a suitable geometry for the first and/or second snap ring to lock in place, the sliding surface can provide for a simple joining of the tracheostoma aid into the connector ring, wherein the tracheostoma aid can be brought into a clamping position with little expenditure of force. It is a further advantage of the funnel-shaped sliding surface that the tracheostoma aid is centered during insertion into the connector ring.

The snap ring is formed as at least partly circumferential ring or a number of ring segments. The first and/or second snap ring may includes a bead-shaped material accumulation. The first and/or second snap ring is adapted in a funnel-shaped manner. In terms of the present disclosure, “funnel-shaped” is to be understood to be a design in particular of a distal surface of the first and/or second snap ring, which is inclined towards the central axis and proximally. The first and/or the second snap ring is designed as protrusion, which extends radially inwards. The first and/or second snap ring includes an annular disk. In terms of the present disclosure, a distal surface and a proximal surface of the first and/or second snap ring is in each case essentially arranged on a plane perpendicular to the central axis of the connector ring. A distal surface and a proximal surface of the first and/or second snap ring are arranged essentially parallel to one another. A distal or a proximal surface of the first and/or second snap ring is in each case essentially arranged on a plane perpendicular to the central axis of the connector ring.

The term “essentially” specifies a tolerance range, which is reasonable for the person of skill in the art under economic and technical aspects, so that the corresponding feature can still be recognized or is realized as such.

In one embodiment, a proximal surface of the first snap ring is arranged on a plane perpendicular to the central axis of the connector ring and a proximal surface of the second snap ring is designed in a funnel-shaped manner. In a further aspect, the proximal surfaces of the first and of the second snap ring are designed in a funnel-shaped manner. It is provided in one embodiment that the proximal side of the first and/or second snap ring is arranged essentially on a plane perpendicular to the central axis of the connector ring. It is provided in one embodiment that an amount of an angle of the proximal side of the first and/or second snap ring to the plane perpendicular to the central axis is smaller than an amount of an angle of the corresponding distal side to the plane perpendicular to the central axis.

A conical connector of a tracheostoma aid can advantageously slide on the funnel-shaped or beveled distal side, respectively, of the first and/or second snap ring and can thereby introduce a force into the connector ring via the distal side, which force deforms said connector ring, such as in order to clamp the tracheostoma aid or to snap it into an undercut. When the tracheostoma aid or the conical connector, respectively, has an undercut, the first or second snap ring can snap back, after the undercut sweeps over the first or second snap ring, and form a positive snap connection with the conical connector. When pulling out the tracheostoma aid, the conical connector introduces a force into the proximal side of the first or second snap ring. When the proximal side of the first and/or second snap ring is arranged essentially on a plane perpendicular to the central axis of the connector ring or an amount of an angle of this proximal side to the plane perpendicular to the central axis is smaller than an amount of an angle of the corresponding distal side to the plane perpendicular to the central axis, the tensile force for removing the tracheostoma aid out of the connector ring is significantly larger than the compressive force for joining the tracheostoma aid into the connector ring.

The first and/or the second snap ring have, in some embodiments, an inner diameter of approximately 11 mm to approximately 25 mm, in other embodiments approximately 21 mm to approximately 24 mm, in other embodiments approximately 11.5 mm, in other embodiments approximately 15 mm, in other embodiments approximately 16 mm, in other embodiments approximately 22 mm, in other embodiments approximately 23 mm.

In one embodiment, the first snap ring includes a first inner diameter. It is provided in one aspect that the first snap ring has a first inner diameter of approximately 16 mm or approximately 23 mm.

In one embodiment, the second snap ring includes a second inner diameter. The second inner diameter is smaller than the first inner diameter. It is provided in one aspect that the second snap ring has a second inner diameter of approximately 15 mm or approximately 22 mm.

In one embodiment, the first inner diameter of the first snap ring is approximately 16 mm and the second inner diameter of the second snap ring is approximately 15 mm. In one embodiment, the first inner diameter of the first snap ring is approximately 23 mm and the second inner diameter of the second snap ring is approximately 22 mm. The difference between first and second inner diameter is preferably approximately 1 mm to approximately 2 mm.

It is provided according to the present disclosure that the first snap ring has a first inner diameter and the second snap ring has a second inner diameter, wherein the first inner diameter is larger than the second inner diameter.

It is provided according to the present disclosure that the first snap ring is arranged distally from the second snap ring. A tracheostoma aid with a smaller outer diameter than the outer diameter of the first snap ring can advantageously be guided past the first snap ring and can be connected to the second snap ring in particular in a positive or non-positive manner.

The second snap ring is arranged proximally from the first snap ring. It is provided in one aspect that the first snap ring is arranged on a distal end of the connector ring. This makes it possible in an advantageous manner that the tracheostoma aid, which is connected to the first snap ring in a clamping or snapping manner, can sit on the second snap ring.

In one embodiment, the connector includes a respiratory protection. The respiratory protection includes at least one web, a mesh and/or a circumferential, ledge. In one embodiment, the respiratory protection includes at least partly circumferential material accumulation on the inner side of the connector ring. The respiratory protection is shaped or configured as ring or annular disk. The respiratory protection advantageously prevents the tracheostoma aid from being pushed through too far. The respiratory protection includes a smaller inner diameter than the first and the second snap ring. The respiratory protection, in some embodiments, is arranged on a proximal end of the connector, and may be on the proximal end of the connector ring. The respiratory protection is shaped or configured in an essentially flat manner with a proximal side of the connecting piece, in the case of the aspect of the connecting piece as base plate. The respiratory protection is material-connected to or is made of the same material as the base plate. The respiratory protection is material-connected to or is made of the same material as the connector ring.

In an exemplary embodiment of the connector, the latter includes a connecting piece. The connecting piece is formed, for example, as base plate of a tape. The connecting piece is arranged, for example, on an adhesive, which is designed as adhesive film. The connecting piece extends radially outwards on the proximal end of the connector ring. The connector ring is shaped or configured in a hollow-cylindrical manner and includes an inner surface. A first snap ring and a second snap ring is formed on the inner surface. The first snap ring is assigned, for example, to a distal end of the connector ring. The first snap ring is shaped or configured, for example, in an annular disk-shaped manner and is arranged in a flush manner with a distal end of the connector ring. The first snap ring has, for example, a first inner diameter of approximately 23 mm. The second snap ring is arranged proximally from the first snap ring and the approximately at half the height in the direction of the central axis of the connector ring. The second snap ring distally includes a sliding surface, which is beveled at an angle of approximately 45° to a central axis of the connector ring. The proximal side of the second snap ring extends, for example, on a plane perpendicular to the central axis of the connector ring. The second snap ring has, for example, a second inner diameter according to DIN EN ISO 5356-1:2015-06 of approximately 22 mm. A respiratory protection is arranged on the proximal end of the connector ring. On the proximal side, the respiratory protection is designed in a flush manner with the connecting piece. The respiratory protection is arranged on the inner surface of the connector ring in an annular disk-shaped manner. The connector ring, the first snap ring, the second snap ring and the connecting piece are made of the same material.

An exemplary first tracheostoma aid according to DIN EN ISO 5356-1:2015-06 with a diameter of approximately 22 mm includes a conical connector, which forms an undercut, which can cooperate with the second snap ring. During an exemplary joining of the first tracheostoma aid into the connector ring of the connector, said first tracheostoma aid is guided past the first snap ring, without interacting therewith, because the first snap ring has a first inner diameter, which is larger than a maximum outer diameter of the conical connector. During the further joining, the conical connector is, for example, guided past the second snap ring, wherein the second snap ring or the connector ring, respectively, is elastically deformed. After the undercut of the conical connector has swept over the second snap ring, the latter essentially snaps back into its original shape, for example, and holds the first tracheostoma aid in the connector in a positive manner. The joining of the first tracheostoma aid is delimited proximally, for example, by the respiratory protection, which is arranged at the height of the connecting piece.

An exemplary second tracheostoma aid has a conical connector with an outer diameter, which is larger than the outer diameter of the first tracheostoma aid. During the joining of the second tracheostoma aid into the connector ring, the conical connector is guided past the first snap ring in such a way that the first snap ring or the connector ring, respectively, is elastically deformed. After an undercut of the conical connector has swept over the first snap ring, the latter essentially snaps back into its original shape and holds the second tracheostoma aid in the connector, for example in a positive manner. The second tracheostoma aid can sit on the second snap ring, whereby an excessive movement of the second tracheostoma aid in the connector is delimited.

An exemplary third tracheostoma aid according to DIN EN ISO 5356-1:2015-6 with a diameter of approximately 22 mm includes a conical connector, which does not form an undercut. During an exemplary joining of the third tracheostoma aid into the connector ring of the connector, said third tracheostoma aid is guided past the first snap ring without interacting therewith, because the first snap ring has a first inner diameter, which is larger than a maximum outer diameter of the conical connector. During the further joining, the conical connector is, for example, guided past the second snap ring, wherein the second snap ring or the connector ring, respectively, is elastically deformed. The joining is ended when the second snap ring forms a frictional resistance with an outer surface of the conical connector, which the user perceives as being sufficient for a clamping. Alternatively, the joining is ended when the force for joining the third tracheostoma aid into the connector ring exceeds approximately 50 N. The joining of the first tracheostoma aid is delimited proximally, for example, by the respiratory protection, which is arranged at the height of the connecting piece.

A tape with an above-described connector is furthermore proposed, wherein the connecting piece is arranged on a distal side of the tape. The tape includes an adhesive, which is applied to the proximal side of the connecting piece. For example, the adhesive is configured as an adhesive film. In one aspect, the adhesive or the adhesive film, respectively, towers above the connecting piece on at least one side, and may on all sides on a plane, in which the adhesive is arranged.

What is furthermore proposed is a tracheal cannula with an above-described connector, wherein a connector ring is arranged on a distal end of a cannula tube. The tracheal cannula can be configured to adapted as inner cannula or outer cannula. The cannula tube can be configured or adapted in a windowed, screened or non-windowed manner.

What is furthermore proposed is a use of an above-described connector for connection to tracheostoma aids with different outer diameters.

### BRIEF DESCRIPTION OF THE DRAWINGS

Further advantageous designs follow from the drawings below. However, the further developments illustrated there are not to be interpreted so as to be limiting, the features described there can instead be combined with one another and with the above-described features to further designs. It is furthermore pointed out that the reference numerals specified in the figure description do not limit the scope of protection of the present disclosure but only refer to the exemplary embodiment shown in the figures. In the following, the same parts or parts with the same function have the same reference numerals, in which:

FIG. 1 schematically depicts shows a perspective view of a tape with a connector;

FIG. 2 schematically depicts shows a view of a longitudinal section of a connector;

FIG. 3 schematically depicts shows a view of a longitudinal section of the connector from FIG. 2 with a first tracheostoma aid;

FIG. 4 schematically depicts shows a view of a longitudinal section of the connector from FIG. 2 with a second tracheostoma aid; and

FIG. 5 schematically depicts shows a tracheal cannula with a connector.

### DETAILED DESCRIPTION OF EMBODIMENTS

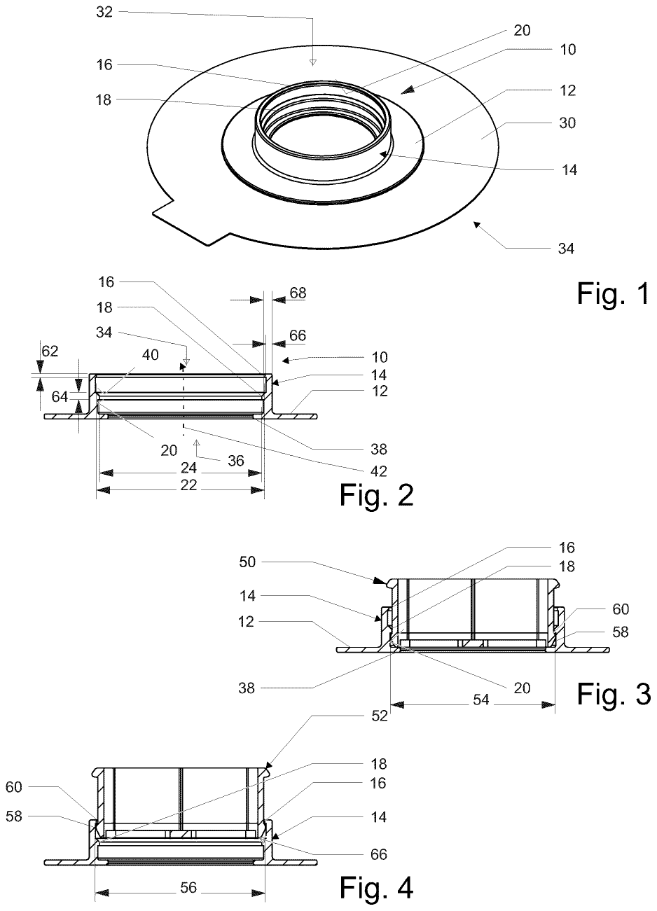

FIG. 1 shows a perspective view of a tape 34 with a connector 10. A connecting piece 12 is formed as base plate of a tape 34. The connecting piece 12 is arranged on an adhesive 30, which is designed as adhesive film with a peelable protective film. A connector ring 14 extends distally from the connecting piece 12. The connector ring 14 is designed in a hollow-cylindrical manner and includes an inner surface 20. A first snap ring 16 and a second snap ring 18 is formed on the inner surface 20. The first snap ring 16 is assigned to a distal end 32 of the connector ring 14. The second snap ring 18 is arranged proximally from the first snap ring 16 and approximately at half the height in the direction of the longitudinal axis of the connector ring.

FIG. 2 shows a view of a longitudinal section of a connector 10 without the tape 34 shown in FIG. 1. The first snap ring 16 is formed in a flush manner with a distal end 32 of the connector ring 14. The first snap ring 16 has a first inner diameter 22 of approximately 23 mm. The first snap ring 16 includes a first maximum material thickness 62. The second snap ring 18 is furthermore formed approximately centrally on the inner surface 20 of the connector ring 14. The second snap ring 18 distally includes a sliding surface 40, which is beveled at an angle of approximately 45° to a central axis 42, wherein the angle is not shown for the sake of clarity. The second snap ring 18 includes a second material thickness 64, wherein the first maximum material thickness 62 is smaller than the second maximum material thickness 64. The second snap ring has a second inner diameter 24 according to DIN EN ISO 5356-1:2015-06 of approximately 22 mm. A respiratory protection 38 is arranged on the proximal end 36 of the connector ring 14. On the proximal side 36, the respiratory protection 38 is formed in a flush manner with the connecting piece 12. The respiratory protection 36 is arranged as annular disk on the inner surface 20 of the connector ring 14. The connector ring 14, the first snap ring 16, the second snap ring 18 and the connecting piece 12 are made of the same material.

FIG. 3 shows a view of a longitudinal section of the connector 10 from FIG. 2 with a first tracheostoma aid 50 according to DIN EN ISO 5356-1:2015-06 with a diameter of approximately 22 mm. The first tracheostoma aid 50 includes a conical connector 58, which forms an undercut 60, which cooperates with the second snap ring 18. During a joining of the first tracheostoma aid 50 into the connector ring 14, said first tracheostoma aid is guided past the first snap ring 16, without interacting therewith, because the first snap ring 16 has a first inner diameter 22, which is shown in FIG. 2, which is larger than a maximum outer diameter 54 of the conical connector 58. During the further joining, the conical connector 58 is guided past the second snap ring 18 in such a way that the second snap ring 18 or the connector ring 14, respectively, is elastically deformed. After the undercut 60 of the conical connector 58 has swept over the second snap ring 18, the latter essentially snaps back into its original shape and holds the first tracheostoma aid 50 in the connector 10 in a positive manner. The joining of the first tracheostoma aid 50 is delimited proximally by means of the respiratory protection 38, which is arranged at the height of the connecting piece 12.

FIG. 4 shows a view of a longitudinal section of the connector 10 from FIG. 2 with a second tracheostoma aid 52. The second tracheostoma aid 52 has a conical connector 58 with an outer diameter 56. The outer diameter 56 of the second tracheostoma aid 52 is larger than the outer diameter 54 of the first tracheostoma aid 50. During the joining of the second tracheostoma aid 52 into the connector ring 14, the conical connector 58 is guided past the first snap ring 16 in such a way that first snap ring 16 or the connector ring 14, respectively, is elastically deformed. After the undercut 60 of the conical connector 58 has swept over the first snap ring 16, the latter essentially snaps back into its original shape and holds the second tracheostoma aid 52 in the connector 10 in a positive manner. The second tracheostoma aid 52 can sit with its distal end 66 on the second snap ring 18, whereby an excessive movement of the second tracheostoma aid 52 in the connector 10 is delimited.

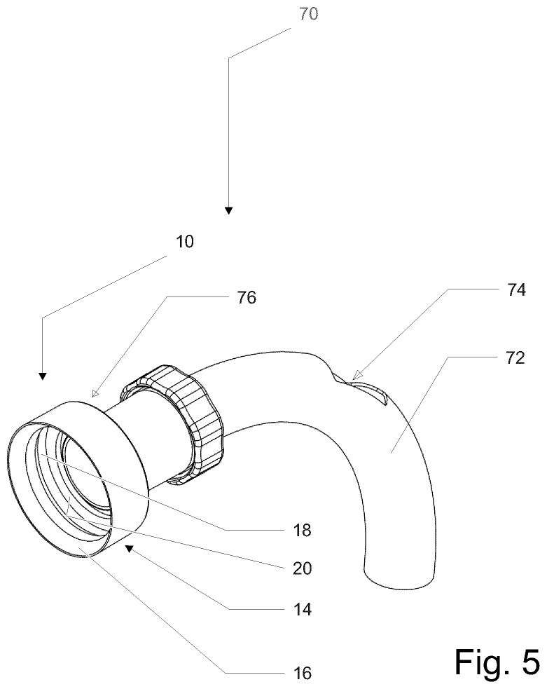

FIG. 5 shows a tracheal cannula 70 with a connector 10. The tracheal cannula 70 is formed as inner cannula and has a windowing 74. As stated in the general description, the tracheal cannula can also be designed in a non-windowed, screened manner and/or as outer cannula. On the distal end of a cannula tube 72, the tracheal cannula 70 has the connector 10 with a connector ring 14. The connector ring 14 includes a first snap ring 16 and at least one second snap ring 18, wherein the first snap ring 16 and the second snap ring 18 are formed on an inner surface 20 of the connector ring 14. The first snap ring 16 has a first inner diameter of approximately 23 mm and the second snap ring has a second inner diameter according to DIN EN ISO 5356-1:2015-06 of approximately 22 mm.

Tracheostoma aids 50, 52 with different nominal dimensions can advantageously be received by means of the proposed connector 10. The connector can in particular be used in an advantageous manner with tracheostoma aids 50 according to DIN EN ISO 5356-1:2015-06 and with non-standardized tracheostoma aids 52.

1. A connector for connection to a tracheostoma aid comprising:
a connector ring, wherein the connector ring having a first snap ring and at least one second snap ring, the first snap ring and the second snap ring are formed on an inner surface of the connector ring, the first snap ring has a first inner diameter and the second snap ring has a second inner diameter, the first inner diameter is larger than the second inner diameter, and the first snap ring and the second snap ring include a bead-shaped material accumulation and are elastically deformable and wherein the first snap ring is arranged distally in a direction away from the body of an individual during normal use of the connector, from the second snap ring.

2. The connector according to claim 1, further comprising:
a connecting piece, which is formed as a base plate.

3. The connector according to claim 1, further comprising:
a cannula tube arranged proximally from the connector ring.

4. The connector according to claim 1, wherein the first snap ring is arranged on a distal end of the connector ring.

5. The connector according to claim 1, wherein the connector ring has a first material thickness between the first snap ring and the second snap ring and has a second material thickness proximally from the second snap ring.

6. The connector according to claim 1, wherein the first snap ring has a first inner diameter of approximately 16 mm or approximately 23 mm.

7. The connector according to claim 1, wherein the second snap ring has a second inner diameter of approximately 15 mm or approximately 22 mm.

8. The connector according to claim 1, further comprising:
a tape; and
a connecting piece arranged on a distal side of the tape.

9. The connector according to claim 1, wherein the tracheostoma aid has different outer diameters.

10. A tracheal cannula comprising:
a cannula tube; and
a connector for connection to the tracheal cannula, the connector having a connector ring arranged on a distal end of the cannula tube, wherein the connector ring having a first snap ring and at least one second snap ring, the first snap ring and the second snap ring are formed on an inner surface of the connector ring, the first snap ring has a first inner diameter and the second snap ring has a second inner diameter, the first inner diameter is larger than the second inner diameter, and the first snap ring and the second snap ring include a bead-shaped material accumulation and are elastically deformable and wherein the first snap ring is arranged distally in a direction away from the body of an individual during normal use of the connector, from the second snap ring.

11. The tracheal cannula according to claim 10, further comprising:
a tape; and
a connecting piece arranged on a distal side of the tape.

12. The tracheal cannula according to claim 10, further comprising:
a cannula tube arranged proximally from the connector ring.

13. The tracheal cannula according to claim 10, wherein the first snap ring is arranged on a distal end of the connector ring.

14. The tracheal cannula according to claim 10, wherein the connector ring has a first material thickness between the first snap ring and the second snap ring and has a second material thickness proximally from the second snap ring.

15. The tracheal cannula according to claim 10, wherein the first snap ring has a first inner diameter of approximately 16 mm or approximately 23 mm.

16. The tracheal cannula according to claim 10, wherein the second snap ring has a second inner diameter of approximately 15 mm or approximately 22 mm.
